(19) United States
(12) Patent Application Publication
(10) Pub. No.: US 2019/0183427 A1
(43) Pub. Date: Jun. 20, 2019

(54) METHOD AND APPARATUS FOR PATIENT BED LOAD CELL SIGNAL MONITORING FOR PATIENT MOVEMENT CLASSIFICATION

(71) Applicant: Hill-Rom Services, Inc., Batesville, IN (US)

(72) Inventors: Yongji FU, Harrison, OH (US); Chunhui ZHAO, Hangzou (CN); Liming LU, Hangzou (CN)

(21) Appl. No.: 16/223,970

(22) Filed: Dec. 18, 2018

Related U.S. Application Data
(60) Provisional application No. 62/607,557, filed on Dec. 19, 2017.

(51) Int. Cl. A61B 5/00; A61B 5/11; A61G 7/05

(52) U.S. Cl. CPC A61B 5/7264; A61B 5/1115; A61B 5/6892; A61G 2203/44; A61B 5/7203; A61B 2562/0252; A61G 7/05

(57) ABSTRACT

A patient support apparatus configured as a sensing device to perform a data-driven classification algorithm to recognize different patient movements by analyzing the real-time signals that are acquired from four load cells installed around the patient support apparatus and performing a probabilistic analysis to discriminate the type of movement based on characterization data.

### PRIORITY CLAIM

This application claims priority under 35 U.S.C. § 119(e) to U.S. Provisional Application No. 62/607,557, filed Dec. 19, 2017, which is expressly incorporated by reference herein.

### BACKGROUND

A patient support apparatus is configured to operate as a sensing device to characterize patient movement by applying a statistical model to real-time data to discriminate the type of movement the patient is making from a predefined set of movements.

Known systems employ various sensors to detect the location of a patient on a patient support apparatus and predict patient activities based on real time signals from load sensors of the patient support apparatus. In general, these systems are limited to classifying the in-bed patient activity into two classes: exiting the bed or not. That is to say, other actions like turning over and sitting up in bed are difficult or impossible to be recognized. Thus these undefined actions will quite possibly be misclassified into exiting due to the high sensitivity. False alarms are therefore generated which will not only create unnecessary distractions but also cause false fatigue on the part of caregivers so that critical alarms are likely to be missed by the staff.

### SUMMARY

The present disclosure includes one or more of the features recited in the appended claims and/or the following features which, alone or in any combination, may comprise patentable subject matter.

According to the present disclosure, using signals form load sensors on a patient support apparatus, a method combining dynamic principal component analysis (DPCA) and Gaussian mixture model (GMM) is used to model various classifications of patient movements. DPCA is utilized as the first step to describe both static and dynamic characteristics of the serial dependent data. Past values of each variable are taken into consideration because of the autocorrelation. Then principal component analysis is used to extract pivotal information from massive signals to improve the precision of the follow-up modeling. A GMM using the Figueiredo-Jain (FJ) algorithm is established with the data in low-dimensional principal component subspace and to represent different classifications. Then final classification is processed based on posterior probability calculated by Bayes Rule. An alarm will be triggered to alert the nursing staff when a dangerous exiting or other actions deserving special attention are detected.

According to one aspect of the present disclosure, a sensing system for detecting and characterizing a patient action comprises a frame, a plurality of load sensors supported from the frame, a patient supporting platform supported from the plurality of load sensors so that the entire load supported on the patient supporting platform is transferred to the plurality of load sensors, and a controller supported on the frame. The controller is electrically coupled to the load sensors and operable to receive a signal from each of the plurality of load sensors with each load sensor signal representative of a load supported by the respective load sensor. The controller includes a processor and a memory device. The memory device includes a non-transitory portion storing instructions that, when executed by the processor, cause the controller to utilize a dynamic principal component analysis and a mixture model to evaluate the temporal distribution of loads sensed by each of the respective load sensors to distinguish the pattern of patient action using real-time monitoring signals to create a model; and monitor the signals from the load sensors to classify the nature of the patient action into a particular one of a plurality of classifications using a probabilistic analysis and modify an operating characteristic of the patient support in response to the particular classification of the patient action.

In some embodiments, the mixture model is operable to draw a probabilistic inference about the likelihood of multiple patient actions in real time to characterize the likelihood of any one of the patient actions and thereby distinguish the likely resulting patient action from multiple patient actions indicated by the load sensor data.

In some embodiments, the dynamic principal component analysis extracts both static and dynamic relations from the signals.

In some embodiments, the mixture model is established by a Gaussian mixture model with a Figueiredo-Jain algorithm.

In some embodiments, median filtering is applied to the load signals to remove the random measurement noise.

In some embodiments, the load signals are normalized to eliminate the effect of the patient's weight.

In some embodiments, the classification is determined by applying Bayes' Theorem.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a perspective view from the foot end on the patient's right of a patient support apparatus;

FIG. 2 is a block diagram of a portion of the electrical system of the patient support apparatus of FIG. 1 used to determine a tare weight of the patient support apparatus;

FIG. 3 is a diagrammatic representation of the positions of a number of load cells relative to the patient support apparatus of FIG. 1;

FIGS. 4A-4F are charts illustrating signals from multiple sensors during specific patient movements;

FIGS. 5A and 5B are charts illustrating the activities of FIGS. 4C and 4E, respectively, as processed by dynamic expansion according to the present disclosure; and

FIG. 6 is flowchart illustrating the steps used to implement the algorithm of the present disclosure.

### DETAILED DESCRIPTION

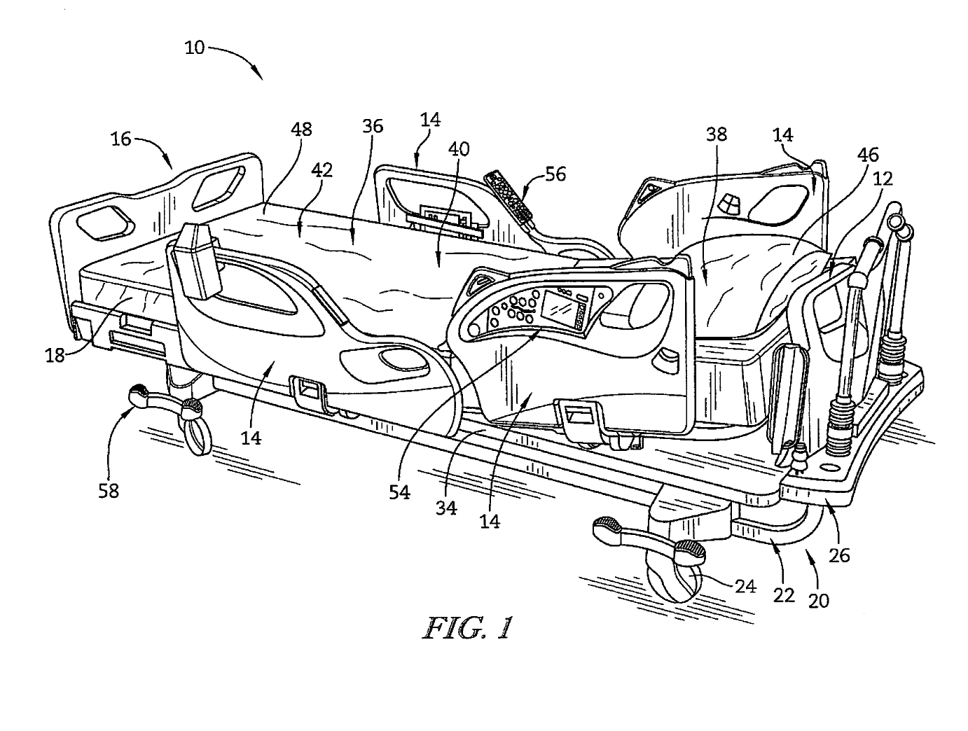

An illustrative patient support apparatus 10 embodied as a hospital bed is shown in FIG. 1. The patient support apparatus 10 of FIG. 1 has a fixed bed frame 20 which includes a stationary base frame 22 with casters 24 and an upper frame 26. The stationary base frame 22 is further coupled to a weigh frame 30 that is mounted via frame member 32a and 32b to an adjustably positionable mattress support frame or deck 34 configured to support a mattress 18. The mattress 18 defines a patient support surface 36 which includes a head section 38, a seat section 40, and a foot section 42. The patient support apparatus 10 further includes a headboard 12 at a head end 46 of the patient support apparatus 10, a footboard 14 at a foot end 48 of the patient support apparatus 10, and a pair of siderails 16 coupled to the upper frame 26 of the patient support apparatus 10. The siderail 16 supports a patient monitoring control panel and/or a mattress position control panel 54. The patient support apparatus 10 is generally configured to adjustably position the mattress support frame 34 relative to the base frame 22.

Conventional structures and devices may be provided to adjustably position the mattress support frame 34, and such conventional structures and devices may include, for example, linkages, drives, and other movement members and devices coupled between base frame 22 and the weigh frame 30, and/or between weigh frame 30 and mattress support frame 34. Control of the position of the mattress support frame 34 and mattress 18 relative to the base frame 22 or weigh frame 30 is provided, for example, by a patient control pendant 56, a mattress position control panel 54, and/or a number of mattress positioning pedals 58. The mattress support frame 34 may, for example, be adjustably positioned in a general incline from the head end 46 to the foot end 48 or vice versa. Additionally, the mattress support frame 34 may be adjustably positioned such that the head section 38 of the patient support surface 36 is positioned between minimum and maximum incline angles, e.g., 0-65 degrees, relative to horizontal or bed flat, and the mattress support frame 34 may also be adjustably positioned such that the seat section 40 of the patient support surface 36 is positioned between minimum and maximum bend angles, e.g., 0-35 degrees, relative to horizontal or bed flat. Those skilled in the art will recognize that the mattress support frame 34 or portions thereof may be adjustably positioned in other orientations, and such other orientations are contemplated by this disclosure.

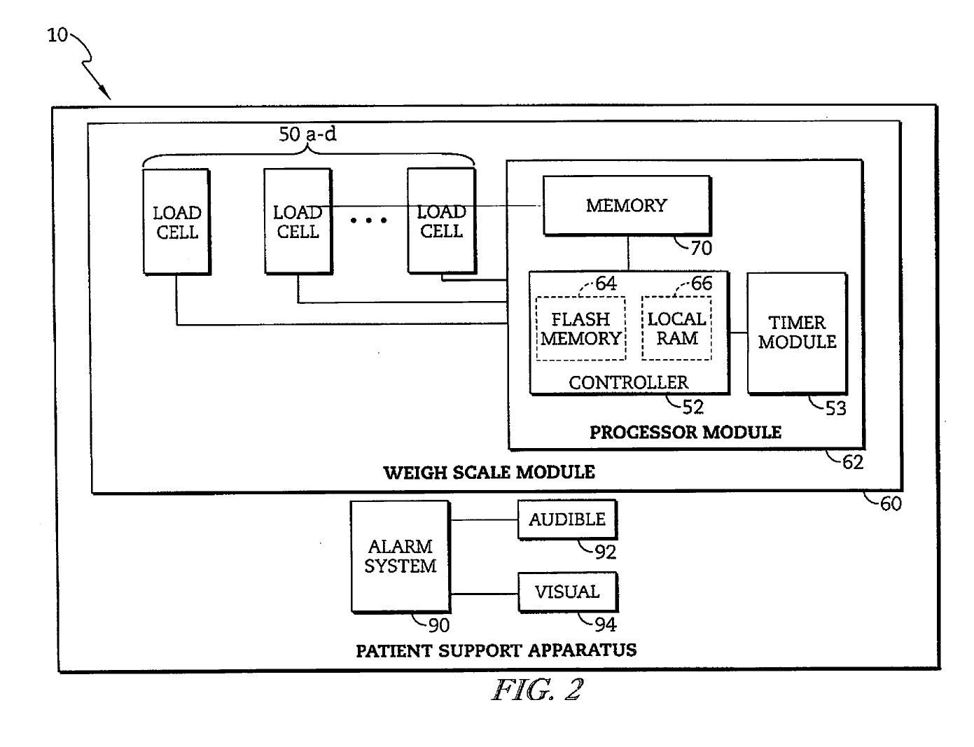

In one illustrative embodiment shown diagrammatically in FIG. 2, the patient support apparatus 10 includes a weigh scale module 60 and an alarm system 90. The weight scale module 60 is configured to determine a plurality set of calibration weights for each of a number of load cells 50 for use in determining a location and an accurate weight of the patient. To determine a weight of a patient supported on the patient support surface 36, the load cells 50 are positioned between the weigh frame 30 and the base frame 22. Each load cell 50 is configured to produce a voltage or current signal indicative of a weight supported by that load cell 50 from the weigh frame 30 relative to the base frame 22. The weigh scale module 60 includes a processor module 62 that is in communication with each of the respective load cells 50. The processor module 62 includes a microprocessor-based controller 52 having a flash memory unit 64 and a local random-access memory (RAM) unit 66. The local RAM unit 66 is utilized by the controller 52 to temporarily store information corresponding to features and functions provided by the patient support apparatus 10. The alarm system 90 is configured to trigger an alarm if the movement of the patient exceeds a predetermined threshold or meets an alarm classification as discussed in further detail below. The alarm may be an audible alarm 92 and/or a visual alarm 94. The visual alarm 94 may be positioned, for example, on the mattress position control panel 54 and/or the patient control pendant 56.

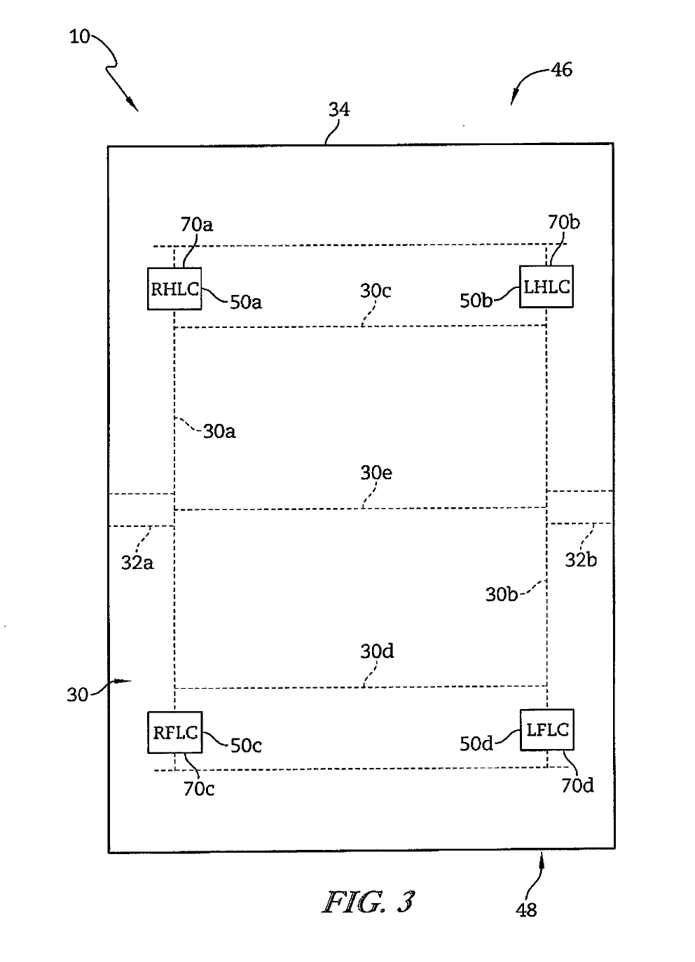

In the illustrated embodiment of FIG. 3, four such load cells 50a-50d are positioned between the weigh frame 30 and the base frame 22; one each near a different corner of the patient support apparatus 10. All four load cells 50a-50d are shown in FIG. 3. Some of the structural components of the patient support apparatus 10 will be designated hereinafter as “right”, “left”, “head” and “foot” from the reference point of an individual lying on the individual's back on the patient support surface 36 with the individual's head oriented toward the head end 46 of the patient support apparatus 10 and the individual's feet oriented toward the foot end 48 of the patient support apparatus 10. For example, the weigh frame 30 illustrated in FIG. 3 includes a head end frame member 30c mounted at one end to one end of a right side weigh frame member 30a and at an opposite end to one end of a left side frame member 30b. Opposite ends of the right side weigh frame member 30a and the left side weigh frame member 30b are mounted to a foot end frame member 30d. A middle weigh frame member 30e is mounted at opposite ends to the right and left side weigh frame members 30a and 30b respectively between the head end and foot end frame members 30c and 30d. The frame member 32a is shown mounted between the right side frame member 30a and the mattress support frame 34, and the frame member 32b is shown mounted between the left side frame member 30b and the mattress support frame 34. It will be understood that other structural support is provided between the weigh frame member 30 and the mattress support frame 34.

A right head load cell (RHLC) 50a is illustratively positioned near the right head end of the patient support apparatus 10 between a base support frame 44a secured to the base 44 near the head end 46 of the patient support apparatus 10 and the junction of the head end frame member 30c and the right side frame member 30a, as shown in the block diagram of FIG. 2. A left head load cell (LHLC) 50b is illustratively positioned near the left head end of the patient support apparatus 10 between the base support frame 44a and the junction of the head end frame member 30c and the left side frame member 30b, as shown in the block diagram of FIG. 3. A right foot load cell (RFLC) 50c is illustratively positioned near the right foot end of the patient support apparatus 10 between a base support frame 44b secured to the base 44 near the foot end 48 of the patient support apparatus 10 and the junction of the foot end frame member 30d and the right side frame member 30a, as shown in the block diagram of FIG. 3. A left foot load cell (LFLC) 50d is illustratively positioned near the left foot end of the patient support apparatus 10 between the base support frame 44b and the junction of the foot end frame member 30d and the left side frame member 30b. In the exemplary embodiment illustrated in FIG. 3, the four corners of the mattress support frame 34 are shown extending beyond the four corners of the weigh frame 30, and hence beyond the positions of the four load cells 50a-50d.

A weight distribution of a load among the plurality of load cells 50a-50d may not be the same depending on sensitivities of each of load cells 50a-50d and a position of the load on the patient support surface 36. Accordingly, a calibration constant for each of the load cells 50a-50d is established to adjust for differences in the load cells 50a-50d in response to the load. Each of the load cells 50a-50d produces a signal indicative of the load supported by that load cell 50. The loads detected by each of the respective load cells 50a-50d are adjusted using a corresponding calibration constant for the respective load cell 50a-50d. In some embodiments, the adjusted loads are then combined to establish the actual weight supported on the patient support apparatus 10. As discussed below, the signals from the load cells 50a-50d may be processed by the processor module 62 to characterize the movement of a patient into one of several classes. Thus, as configured, the bed 10 is operable as a sensor system for detecting and characterizing patient movement to provide information about the patient movement to a user either through an alarm or other communication method.

For example, six movements that patients may frequently take are considered by theprocessor module 62 and, when a particular movement is detected with specificity, the processor module 62 will characterize the particular movement and act on that characterization according to pre-defined protocols. The movements characterized in the illustrative embodiment include the patient exiting from the bed 10, turning over from right to left, turning over from left to right, stretching out for something (e.g. reaching for a glass of water on a nearby table), sitting up and lying down. The first five actions begin with the patients lying flat on the bed 10, while the last one starts by sitting on the bed 10. These movements are designate as one of an action class G1-G6, where: G1 is patient exiting, G2 is turning over from right to left, G3 is turning over from left to right, G4 is reaching, G5 is sitting up, and G6 is lying down.

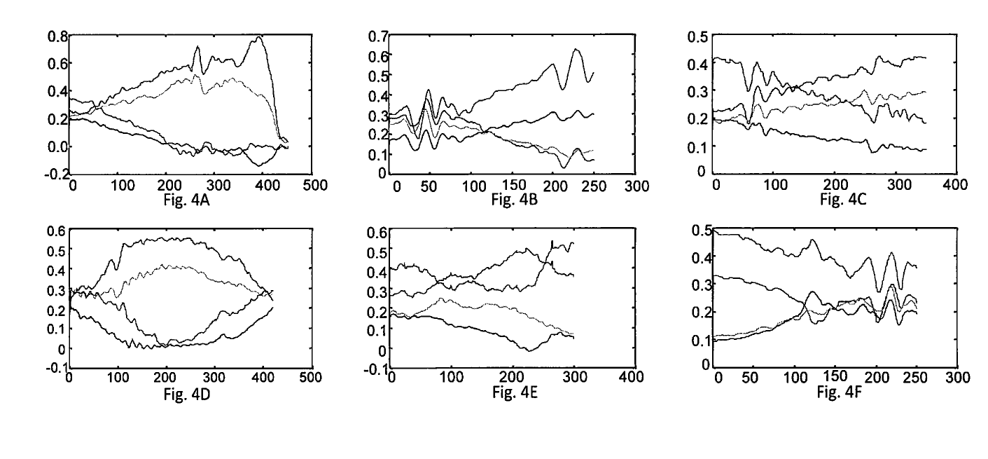

Referring to the flowchart shown in FIG. 4, the system of the present disclosure utilizes an algorithm 98 that includes characterization sampling at step 100, signal processing at step 102, modeling at step 104, monitoring patient activity at step 106, and providing output at step 112.

At the characterization sampling step 100, test subjects are placed on the bed 10 and prompted to perform the various movements while the signals from each of the load cells 50a-50d is monitored. Appropriate sampling is implemented so that variations in the speed of movement and the manner in which various individuals execute the movements are monitored to provide a statistically significant sample over a range of patient demographics.

Once the characterization sampling step 100 is completed, the collected data is subjected to signal processing at step 102 to simplify the data analysis. For example, the initial signal is subjected to a tare analysis to remove any structure offsets applied to the load cells 50a-50d such as by the weight of components of the bed 10. Also, median filtering is applied to the signals to remove any random measurement noise. The signals are also adjusted to a notionally common scale according to Equation (1):

$${x}_{i} = \frac{{P}_{i}}{G} \\ ( i = 1 , 2 , 3 , 4 ) \qquad ( 1 )$$

where P₁ through P₄ are referred to herein as filtered force signal produced by the corresponding load cells 50a-50d for simplicity. G is the total patient weight and is calculated by G=P₁+P₂+P₃+P₄. xᵢ is the normalized data that contains all the values falling between 0 and 1, which has eliminated the effect of patient's weight. Thus, at each time interval t each load cell 50a-50d signal will have been normalized to a respective unit-less proportion of the total weight detected by all four of the load cells 50a-50d.

This signal data is then characterized at step 104 in two sub-steps 108 and 110. In the illustrative embodiment, step 108 includes signal characteristic by dynamic principle component analysis (DPCA) with each of the signals of the four load cells 50a-50d being subjected to the DCPA. At step 110, a Gaussian mixture model is built using an Figueirdo-Jain (FJ) algorithm.

The data set from step 102 is deemed to contain redundant information resulting from the constraining relations between the signals of the respective load cells 50a-50d. In this embodiment, extracting key variables and omitting uninformative variables is preferred. Principal component analysis (PCA) is one of the most popular dimension reduction methods. However, for the dynamic system of signal acquisition in bed 10, the current value of each load cell 50a-50d partly depends on the past values due to the autocorrelation as described in W. Ku, R. H. Storer, C. Georgakis, “Disturbance detection and isolation by dynamic principal component analysis.” Chemometrics and intelligent laboratory systems, 30.1 (1995), 179-196, which is incorporated by reference herein for the discussion of autocorrelation of related sensor signals. Conventional PCA can only demonstrate a linear static approximation. Therefore an extended method DPCA is performed in order to reveal both static and dynamic relations.

If we denote X(N×J) as the data matrix that is composed of the set of continuous load cell signal values belonging to a specific action, in which J is equal to 4 (the number of load cells 50a-50d) and N rests with the duration of the action. The vector of the measurement variables at time t is represented as xₜ (4×1). The vector mentioned in this disclosure is a column vector when there is no special statement.

$$X = [ \begin{matrix}{x}_{11} & {x}_{12} & {x}_{13} & {x}_{14} \\ {x}_{21} & {x}_{22} & {x}_{23} & {x}_{24} \\ ⋮ & ⋮ & & ⋮ \\ {x}_{n 1} & {x}_{n 2} & {x}_{n 3} & {x}_{n 4}\end{matrix} ] = [ \begin{matrix}{x}_{1}^{T} \\ {x}_{2}^{T} \\ ⋮ \\ {x}_{n}^{T}\end{matrix} ] \qquad ( 2 )$$

To explain the autocorrelation property in the static PCA model, take xₜ₋₁ into consideration at time k at least. In other words, in addition to the present one, the variable will be extended with L previous values. The vector xₜ (4×1) is replaced by an augmented vector tₜ(4(L+1)×1), and thus a new data matrix X_{D} including dynamic behavior of an action in the bed 10 is reconstructed.

$${x}_{D} = [ \begin{matrix}{x}_{t}^{T} & {x}_{t - 1}^{T} & … & {x}_{t - L}^{T} \\ {x}_{t - 1}^{T} & {x}_{t - 2}^{T} & … & {x}_{t - L - 1}^{T} \\ ⋮ & ⋮ & & ⋮ \\ {x}_{t - n + L}^{T} & {x}_{t - n + L - 1}^{T} & … & {x}_{t - n}^{T}\end{matrix} ] = [ \begin{matrix}{t}_{t}^{T} \\ {t}_{t - 1}^{T} \\ ⋮ \\ {t}_{t - n + L}^{T}\end{matrix} ] \qquad ( 3 )$$

By means of orthogonal transformation, linearly uncorrelated information in the corresponding principal component subspace (PCS) is expected to be extracted from X_{D}. The transformation is defined by:

Y=X_{D}U (4)

where Y is the output matrix that is composed of uncorrelated variables, in which most of the information of X_{D} is retained. Taking advantage of some conclusions from linear algebra, u is the transformation matrix consisting of the eigenvectors calculated from S, which is presented by:

S=UΛU^{T} (5)

where S(4(L+1)×4(L+1)) denotes the covariance matrix of X_{D}. Λ is a diagonal matrix with elements being the eigenvalues (λ₁, λ₂, . . . λₖ) in descending order of S and is the covariance matrix of the principal components.

The DPCA transformation is defined so that the first principal component corresponding to the largest eigenvalue accounts for the variability of the data as much as possible, while the succeeding component has the second largest eigenvalue, and so on. It's worth noting that only the first n (n<=k) eigenvalues are non-zero in A due to the redundancy mentioned above, and their corresponding eigenvectors can be used to establish the DPCA model. This has explained the reason why PCA is regarded as an effective method in dimensional reduction. What's more, usually only m (m<n) principal components are chosen in practical application so as to simplify the data structure. Specifically, the parameter m can be determined by the cumulative contribution rate calculated by

$${\sum }_{i = 1}^{m} {λ}_{i} / {\sum }_{i = 1}^{n} {λ}_{i} .$$

Then the final output Y(N′×m) of dynamic PCA model is given by:

Y=X_{D}W (6)

where W(k×m) is transformation matrix formed by the m chosen eigenvectors.

It has been proved that mixture models are often regarded as better choice to represent arbitrarily class-conditional probability density functions (PDFs) than traditional models. And the Gaussian mixture model, as a powerful and flexible probabilistic method, has been successfully used to multivariate and univariate data especially in the area of statistical pattern recognition, where it demonstrates a superior performance in classifying the continuous process data collected from different patterns. Consequently, GMM is adopted here to learn the probability distribution of each action with the data Y gained by the dynamic PCA discussed above.

Let Y=[y₁^{T} y₂^{T} . . . y_{N′}^{T}]^{T} with y of dimension m×1. According to the underlying assumption of GMM, signals from different actions follow different Gaussian distributions with distinct covariances and means. For the signal vector y, it comes from several possible Gaussian distributions and GMM is the mixture of these Gaussian components which can thus be defined as:

$$p ( y \vert θ ) = {\sum }_{i = 1}^{M} {P}_{i} p ( y \vert {θ}_{i} ) \qquad ( 7 )$$

where M is the number of mixtures. Pᵢ is the prior probability, or sometimes called weight of the ith Gaussian component. Pᵢ must satisfy a constraint that

$${\sum }_{i = 1}^{M} {P}_{i} = 1 ,$$

Pᵢ≥0, i=1, 2, . . . , M. p(y|θᵢ) denotes the conditional probability density function, in which θᵢ is the set of parameters defining that corresponding component that consists of 2 elements: the covariance matrix Σᵢ and the mean vector μᵢ. All the components are assumed to follows normal distributions, and hence p(y|θᵢ) is given by:

$$\begin{matrix}p ( y \vert {θ}_{i} ) = p ( y \vert {μ}_{i} , {Σ}_{i} ) \\ = \frac{1}{{( 2 π )}^{m / 2} \vert {Σ}_{i} {\vert}^{1 / 2}} exp [ - \frac{1}{2} {( y - {μ}_{i} )}^{T} {{Σ}_{i}}^{- 1} ( y - {μ}_{i} ) ]\end{matrix} \qquad ( 8 )$$

The accuracy of the model is directly related to the component number M. That is to say, if M is small, GMM may not meet the demanded accuracy of modeling. However, the computational complexity will increase dramatically when M is increasing. The selection of M is actually a trade-off between the precision of the model and amount of computation. The conventional GMM method always sets M by experience and M is fixed in different classes.

In this disclosure, M, as well as other parameters (P,θ) are estimated by Figueiredo-Jain (FJ) algorithm which is proposed based on the standard expectation and maximization algorithm. E-step and M-step are alternately applied to yield the sequence of estimates until the convergence criterion is met. The procedure is iterated as follows:

In the E-step,

$${P}^{r} ( {θ}_{i} \vert {y}_{j} ) = \frac{{P}_{i}^{r} p ( {y}_{j} \vert {μ}_{i}^{r} , {Σ}_{i}^{r} )}{{\sum }_{m = 1}^{K} {p}_{m}^{r} p ( {y}_{j} \vert {μ}_{m}^{r} , {Σ}_{m}^{r} )} \qquad ( 9 )$$

where P^{r} (θᵢ|yⱼ) is the posterior probability of the jth training sample produced by the ith Gaussian component. Pᵢ^{r} is the corresponding prior probability. The superscript r denotes the rth iteration. And K is the possible number of Gaussian component that may change with the iteration times.

In the M-step, the parameter estimates are updated according to:

$$\begin{matrix}{μ}_{i}^{r + 1} = \frac{{\sum }_{j = 1}^{{N}^{′}} {P}^{r} ( {θ}_{i} \vert {y}_{j} ) {y}_{j}}{{\sum }_{j = 1}^{{N}^{′}} {P}^{r} ( {θ}_{i} \vert {y}_{j} )} & ( 10 ) \\ {Σ}_{i}^{r + 1} = \frac{{\sum }_{j = 1}^{{N}^{′}} {P}^{r} ( {θ}_{i} \vert {y}_{j} ) ( {y}_{j} - {μ}_{i}^{r + 1} ) {( {y}_{j} - {μ}_{i}^{r + 1} )}^{T}}{{\sum }_{j = 1}^{{N}^{′}} {P}^{r} ( {θ}_{i} \vert {y}_{j} )} & ( 11 ) \\ {P}_{i}^{r + 1} = \frac{max \{ 0 , ( {\sum }_{j = 1}^{{N}^{′}} {P}^{r} ( {θ}_{i} \vert {y}_{j} ) ) - \frac{V}{2} \}}{{\sum }_{i = 1}^{K} max \{ 0 , ( {\sum }_{j = 1}^{{N}^{′}} {P}^{r} ( {θ}_{i} \vert {y}_{j} ) ) - \frac{V}{2} \}} & ( 12 )\end{matrix}$$

where μᵢ^{r+1}, Σᵢ^{r+1}, and Pᵢ^{r+1} are respectively the mean, covariance, and prior probability representing the ith Gaussian component at the (r+1)th iteration that update the parameters got from rth iteration. The covariance matrix Σ has (m²+m)/2 parameters as it owns a symmetric structure. And the mean vector μ has m parameters. V is the total number of parameters defining each Gaussian component and is equal to (m²+3m)/2 in this paper.

Compared with the standard methods, the FJ algorithm is much less initialization dependent, which is able to get the optimal component number of each class by only retaining the non-zero-probability component during the iteration procedure. No prior knowledge is required for determining the specific number. The parameter K is usually set relatively large at first (e.g. 10) as it will decrease after iteration and the final value of K is to assigned to the parameter M to form the monitoring model.

Step 106 is applied when a patient is in the bed 10 and the signals from the load cells 50a-50d are monitored so that when a patient movement is detected, it may be classified into one of the six action classes (G₁ through G₆). For consistency, observations (continuous signals for a period of time) are denoted by having been processed by the dynamic PCA in advance as Y=[y₁^{T} y₂^{T} . . . y_{N′}^{T}]^{T}, where yₜ is a m-dimensional vector representing the expanded load cell signals at sample point t, N′ is the total sample number that is related to the duration of the observation.

Taking a single vector y as an example, Bayes' theorem is adopted to determine its possible class, which is defined by the following expression:

$$P ( {G}_{i} \vert y ) = \frac{P ( y \vert {G}_{i} ) P ( {G}_{i} )}{{\sum }_{j = 1}^{6} P ( y \vert {G}_{j} ) P ( {G}_{j} )} , \\ i = 1 , 2 , … , 6 \qquad ( 13 )$$

where P(Gᵢ|y) is the conditional probability of the event that y belongs to class Gᵢ. And P(y|Gᵢ) denotes the probability of observing y when the class Gᵢ is given. P(Gᵢ) is the prior probability that is usually got by experience and large amount of characterization data at step 100.

Considering the fact that the denominator of Eq. (13) is used for normalization as it stays the same no matter which class is chosen, we here omit it and the discrimination function, indicated by h(y), can be deduced by combining the Eqs. (8) and (13). The formula is as follows:

$$\begin{matrix}{h}_{i} ( y ) = - \frac{1}{2} {( y - {μ}_{i} )}^{T} {{Σ}_{i}}^{- 1} ( y - {μ}_{i} ) - \frac{1}{2} ln \vert {Σ}_{i} \vert \\ - \frac{1}{2} ln 2 π + ln P ( {G}_{i} )\end{matrix} \qquad ( 14 )$$

where μᵢ and Σᵢ are the parameters acquired beforehand and formed the known model of action class Gi.

Considering the assumption that the prior probabilities P(G₁) through P(G₆) are equal in the interest of simplicity, h(y) can be further simplified and the final classification rule is expressed according to the maximum a posterior (MAP) criterion. y will be classified to class Gₖ, if:

$$\begin{matrix}{h}_{k} ( y ) = {max}_{1 \leq i \leq 6} {h}_{i} ( y ) \\ = {max}_{1 \leq i \leq 6} \{ - \frac{1}{2} {( y - {μ}_{i} )}^{T} {{Σ}_{i}}^{- 1} ( y - {μ}_{i} ) - \frac{1}{2} ln \vert {Σ}_{i} \vert \}\end{matrix} \qquad ( 15 )$$

Considering that the human action in general lasts for a period of time, the discrimination function hence needs to be modified to fit the sequence data Y(N′×m). Y will be classified to class Gₖ, if:

$$\begin{matrix}{H}_{k} ( y ) = \frac{1}{{N}^{′}} {\sum }_{j = 1}^{{N}^{′}} {h}_{k} ( {y}_{j} ) & ( 16 ) \\ = {max}_{1 \leq i \leq 6} \{ \frac{1}{{N}^{′}} {\sum }_{j = 1}^{{N}^{′}} [ - \frac{1}{2} {( {y}_{j} - {μ}_{i} )}^{T} {{Σ}_{i}}^{- 1} ( {y}_{j} - {μ}_{i} ) - \frac{1}{2} ln \vert {Σ}_{i} \vert ] \} & \end{matrix}$$

In the illustrative embodiment, the determination of the particular classification as G1-G6 is tested for probability of the determination being a true condition and if the error is sufficiently small, the movement is characterized in the particular classification such that the processor module 62 signals that movement to the alarm system 90 so that a user, such as a nurse, may be notified of the movement and take corrective action. Various corrective actions may be implemented by the user/caregiver/nurse or other systems on the bed 10 may be signaled to initiate a corrective action. For example, portions of the bed 10 may be moved automatically to make the indicated movement easier for the patient.

### Example

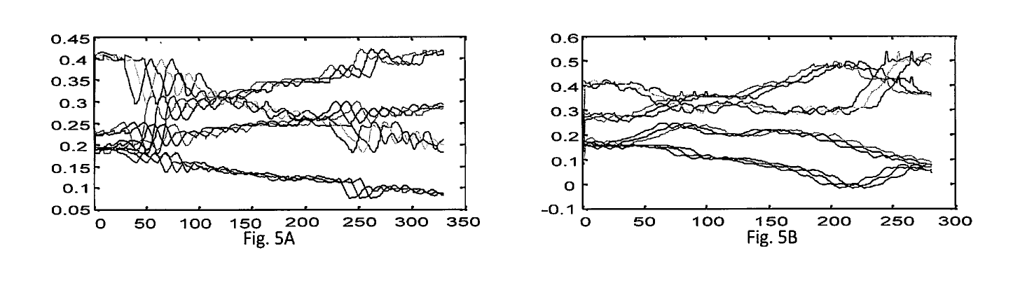

As mentioned earlier, 6 kinds of patient activities G1-G6 related to use of the bed 10 are considered in the present disclosure, exiting from the bed (GI), turning over from right to left (G2), turning over from left to right (G3), stretching out for something (G4), sitting up (G5) and lying down (G6). Experimental data was provided by 10 adults age of 22 through 30 and weigh between 45 to 80 kilograms. For the six actions mentioned before, the number of samples for each action is: G1=151; G2=276; G3=316; G4=149; G5=274; and G6=292. The corresponding load cell signals of each activity after filtering, initializing, and normalizing are shown in FIGS. 5A-5F, respectively. In the illustrative example, the sampling frequency was adjusted to 100 Hz. The abscissa denotes the sample number ordered by sampling time, and the ordinate is the output of value. The fluctuations show the load cells 50a-50d four respective signals' response to the experimental patient's movement. The proposed method tends to capture the intrinsic structure of each class.

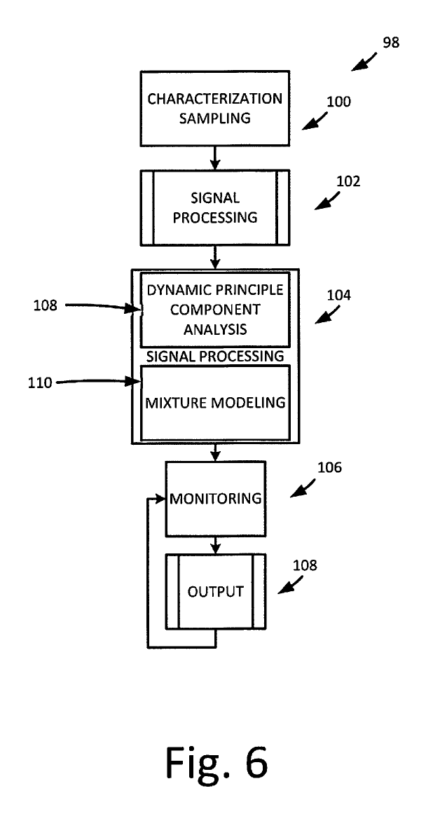

Two movements G3 and G5 are compared in FIGS. 6A and 6B which illustrates these two kinds of signals shown in FIGS. 5C and 5E, respectively, processed by the dynamic expansion as it's expressed in Eq. (3). The parameter L is set to be 2, which means for each load cell 50a-50d, the last 2 signal values are taken into account at the current sampling time, with the purpose of considering the relation within each respective signal. Accordingly, the single observation vector is 12-dimensional instead of the original 4-dimensional. In addition, PCA is further applied to extract the first 4 directions that are most significant to discriminate between different human activities, while the uninformative variables are omitted at the same time.

Here, a conventional GMM classification method is also developed for comparison with the proposed method to evaluating the effects of DPCA. The conventional method builds the monitoring model directly using the original 4-dimensional signal data without DPCA. In other words, the relations between and within signals are not involved in modeling.

The accuracy of classification is used as an indicator so as to evaluate the performance, and the comparison results are shown in Table I, where for convenience, DPCA+GMM denotes the proposed method and GMM denotes the conventional method. Moreover, the specific classification results are summarized in Table II, in which the number in bold style shows the correct classification number.

TABLE I
CLASSIFICATION ACCURACY COMPARISON BETWEEN
THE TWO METHODS

| | Classification Accuracy (%) | | | | | |
|---|---|---|---|---|---|---|
| | | | | Stretch | | |
| Method | | Turn over | Turn over | out for | | Lie |
| Class | Exit | to left | to right | sth. | Sit up | down |
| Proposed method: | 60.26 | 80.43 | 67.41 | 88.59 | 75.55 | 75.00 |
| DPCA + GMM | | | | | | |
| Conventional | 13.91 | 53.25 | 12.03 | 89.93 | 15.69 | 75.34 |
| method: GMM | | | | | | |

TABLE II
CLASSIFICATION RESULTS USING THE TWO METHODS

| | samples | | | | | | | | | | | |
|---|---|---|---|---|---|---|---|---|---|---|---|---|
| | | | Turn over | | Turn over | | Stretch out | | | | | |
| | Exit | | to left | | to right | | for sth. | | Sit up | | Lie down | |
| Classification | DPCA + | | DPCA + | | DPCA + | | DPCA + | | DPCA + | | DPCA + | |
| results | GMM | GMM | GMM | GMM | GMM | GMM | GMM | GMM | GMM | GMM | GMM | GMM |
| Exit | 91 | 21 | 10 | 3 | 23 | 3 | 7 | 92 | 10 | 12 | 10 | 20 |
| Turn over to left | 4 | 1 | 222 | 147 | 4 | 0 | 3 | 44 | 23 | 26 | 20 | 58 |
| Turn over to right | 10 | 0 | 25 | 5 | 213 | 38 | 41 | 164 | 12 | 25 | 15 | 84 |
| Stretch out | 2 | 2 | 0 | 1 | 12 | 7 | 132 | 134 | 1 | 1 | 2 | 4 |
| Sit up | 5 | 0 | 23 | 12 | 13 | 14 | 5 | 43 | 207 | 43 | 21 | 162 |
| Lie down | 11 | 0 | 17 | 5 | 11 | 22 | 12 | 22 | 22 | 21 | 219 | 222 |

It can be seen from the result tables that confusions are likely to happen among exiting, turning over from left to right and stretching. It can be partly explained by the experimental conditions in the lab in that a bedside table is positioned on the right. A test subject is deemed to complete a stretching action when he has got some documents or drinks from the table. In addition, most test subjects participating in the research are accustomed to exiting the bed 10 from the right side. The above three kinds of actions thus share a similar trend of signals for the first half of the movement as they are all moving towards the right side. In that case, the probability of each class is approximately equal. The deficiency is more obvious in the conventional method, and the disclosed method has significantly improved it.

It is clear that the disclosed method has illustrated a superior classification performance, especially for the actions deserving more attention including exiting and turning over. The conventional methods only achieve slightly higher accuracy in the classes of stretching and lying down. To be more specific, for the proposed method, 213 samples are correctly classified among a total of 316 samples when the volunteer is turning over from right to left, reaching accuracy as high as 67.41%. Nevertheless, the accuracy is down to 12.03% with the conventional method, in which only 38 samples are successfully distinguished. As for the exiting action, the proposed method achieves a correct number of 91, compared with 21 from the other method. The conventional method has greater possibility in classifying an unauthorized exit into stretching or any other actions, which may put the patient in a dangerous situation.

The disclosed approach is a data-driven human activity classification method based on load cell signals for a hospital bed. By combining the two multivariate statistical analysis algorithms, dynamic PCA and GMM, dynamic correlations are taken into account, which has significantly increased the precision of modeling. The final classification process is implemented by Bayes' theorem to calculate the probability of each class. In comparison with the conventional method, the disclosed method turns out to be more effective in recognizing the six kinds of common actions G1-G6 for patients on the hospital bed.

Although this disclosure refers to specific embodiments, it will be understood by those skilled in the art that various changes in form and detail may be made without departing from the subject matter set forth in the accompanying claims.

1. A sensing system for detecting and characterizing a patient action comprising
a frame,
a plurality of load sensors supported from the frame,
a patient supporting platform supported from the plurality of load sensors so that the entire load supported on the patient supporting platform is transferred to the plurality of load sensors,
a controller supported on the frame, the controller electrically coupled to the load sensors and operable to receive a signal from each of the plurality of load sensors with each load sensor signal representative of a load supported by the respective load sensor, the controller including a processor and a memory device, the memory device including a non-transitory portion storing instructions that, when executed by the processor, cause the controller to:
utilize a dynamic principal component analysis and a mixture model to evaluate the temporal distribution of loads sensed by each of the respective load sensors to distinguish the pattern of patient action using real-time monitoring signals to create a model; and
monitor the signals from the load sensors to classify the nature of the patient action into a particular one of a plurality of classifications using a probabilistic analysis and modify an operating characteristic of the patient support in response to the particular classification of the patient action.

2. The sensing system of claim 1, wherein the mixture model is operable to draw a probabilistic inference about the likelihood of multiple patient actions in real time to characterize the likelihood of any one of the patient actions and thereby distinguish the likely resulting patient action from multiple patient actions indicated by the load sensor data.

3. The sensing system of claim 1, wherein the dynamic principal component analysis extracts both static and dynamic relations from the signals.

4. The sensing system of claim 1, wherein the mixture model is established by a Gaussian mixture model with a Figueiredo-Jain algorithm.

5. The sensing system of claim 1, wherein median filtering is applied to the load signals to remove the random measurement noise.

6. The sensing system of claim 1, wherein the load signals are normalized to eliminate the effect of the patient's weight.

7. The sensing system of claim 1, wherein the classification is determined by applying Bayes' Theorem.
